(19) United States
(12) Patent Application Publication
(10) Pub. No.: US 2022/0233872 A1
(43) Pub. Date: Jul. 28, 2022

(54) Percutaneous Implantable Pulse Generator

(71) Applicant: Uro Medical Corporation, Boca Raton, FL (US)

(72) Inventors: Laura Grace Perryman, Miami Beach, FL (US); Richard Lebaron, Surprise, AZ (US); Patrick Larson, Boca Raton, FL (US)

(21) Appl. No.: 17/643,218

(22) Filed: Dec. 8, 2021

Related U.S. Application Data
(60) Provisional application No. 63/122,597, filed on Dec. 8, 2020.

(51) Int. Cl. A61N 1/372; H04B 5/00; H01Q 1/27; H01Q 9/16

(52) U.S. Cl. CPC A61N 1/37229; H01Q 9/16; H01Q 1/273; H04B 5/0037

(57) ABSTRACT

A medical apparatus includes a tubular shaped enclosure configured for implantation into a tissue medium; a receiver array with a multitude of receiver elements housed within the enclosure attached to the associated electronics via a flexible circuit board construction, wherein the receiver array is configured to receive one or more electromagnetic input signals of a combination of both power and data from an external transmitter via non-inductive coupling energy transfer, wherein the receiver array is composed of multiple receiver elements, wherein each receiver element within the receiver array includes an electrically small antenna and one or more processor circuits connected to the port of the antenna on the same physical substrate, wherein the receiver array and associated flexible circuit board are directly attached to two or more electrodes that are in direct contact with biological tissue for the purpose of transmitting stimulation pulses to tissue.

The present application claims the benefit of U.S. Provisional Patent Application Ser. No. 63/122,597, filed Dec. 8, 2021, which application is incorporated herein by reference in its entirety.

### FIELD OF THE INVENTION

This disclosure generally relates to systems and methods of operation of an implantable medical apparatus that delivers electrical impulses to excitable tissue utilizing an array of receiver elements in various configurations with or without on board storage capacity over extended periods of time to apply the signals to tissue. The receiver array receives data transmitted from an external device, and transmits data to an external device. The receiver array elements may be formed from dipole antennas embedded within a flexible substrate directly attached to electrodes and associated circuitry for signal routing and power storage.

### BACKGROUND OF THE INVENTION

Electrical devices implanted in the human body can modulate excitable tissue in order to produce medically therapeutic effects. Typically, the power source for such a device is a battery housed in a hermetically sealed enclosure. In some implementations, the battery can be recharged wirelessly by magnetic induction from an external transmitter. Data signals can also be sent to the device via the inductive-coupling pathway.

Implantable microstimulators have been disclosed in the art providing various types of therapy by stimulating or blocking electric signals. In surrounding nerves that are in proximity to the electrode elements of the device. Typically, such devices consist of a housing, electronics circuitry, and a connector to a physically separate electrode array that comprises of wires enclosed in a catheter type housing to route the signals to electrodes found at the end of the wires. The most efficient microstimulators mitigate losses of energy from the power source to the electrodes and transfer an optimal amount of electrical current to the spaced electrodes in the tissue.

This disclosure presents a similar device, however, the power source in the device is not based on battery technology. Rather, miniature storage banks are employed, which can be distributed across different locations within the device. An external transmitter provides wireless recharging power via a first radio frequency (RF) signal in a frequency band from about 200 MHz to 10 GHz, wherein the RF signal is conveyed via polarized electric field transmission, not via magnetic induction. The external transmitter can also send and receive data signals via additional RF signals in the band. The goal of miniaturization of energy storage is to reduce the size of the implantable device such that the device can be implanted into tissue in a minimally invasive manner. In some embodiments, the device is capable of passing through the inner lumen of a needle-sized introducer. The device is recharged by electromagnetic power that is received through the skin from an external transmitter to a receiver array embedded within the implantable device. The implantable medical device can include a pulse generator and other electrical subsystems, where all components are housed in the same enclosure.

Certain legacy technologies in this field have utilized single-element, half-wavelength dipole antennas as a compromise to facilitate the implantable device fitting into a miniature enclosure that is constrained by a small diameter. However, such dipole antennas only perform well when the dipole has optimal alignment to the incident electromagnetic waves sent by the external transmitter. A single, half-wavelength dipole antenna has several drawbacks when implemented in real-world scenarios. These limitations include:

Angle relative to face of transmitting antenna phase alignment issues: When the half-wavelength dipole antenna is angled away from the face of the transmitting antenna, the half-wavelength receiver dipole is subject to destructive interference due to the reach of the antenna arms into fields that are out of phase.

Angle relative to polarization of transmitting antenna (Azimuth angle): Similarly, a half-wavelength dipole antenna has high sensitivity to azimuth angular alignment relative to the external transmitting antenna. Optimal transmission is only achieved when the polarization of the half-wavelength dipole antenna is closely aligned with the polarization of the external transmitting antenna. When a half-wavelength dipole antenna is rotated out of alignment, the received power decreases rapidly with the angle.

Number of extraction circuits: A half-wavelength dipole antenna has inherently only one port at which RF power can be extracted and processed. Extraction of power or information from an input signal is limited by the physics of the half-wavelength dipole antenna. For such an antenna residing in a medium such body tissue, several factors can hinder the performance of a single port antenna.

Longitudinal alignment relative to transmitting antenna: A half-wavelength dipole antenna has high sensitivity to longitudinal alignment with the external transmitting antenna. Optimal transmission is only achieved when the feed port of the half-wavelength dipole antenna is closely aligned with the center of the external transmitting antenna. When a half-wavelength dipole antenna is misaligned, the received power decreases rapidly with the degree of misalignment.

Size of antenna aperture: A half-wavelength dipole antenna is inherently constrained to have an antenna aperture defined by the size of the halfwave dipole in the medium. Typically, the half-power roll-off in the medium (tissue) is on the order of 1/10^{th} of the wavelength, which can be problematic in real-world implantation scenarios.

Geometrical linearity of antenna: The performance of a half-wavelength dipole antenna decreases as the antenna arms become curved or bent. If the curvature occurs in the azimuth angle, becoming out of polarization, a portion of the dipole intercepts less field. Whereas if the curvature is either away or toward the face of the transmitting antenna, a portion of the dipole reaches into a zone having the same phase as the other antenna arm. This reduces the driving potential across the antenna port. Thus, for optimal performance of a half-wave dipole, it is essential to maintain geometrical linearity of the antenna, however in real-world implantation scenarios, it is seldom possible to fixate the antenna in such a way as to maintain geometrical linearity.

Specific absorption rate (SAR): RF electric fields are absorbed by any lossy medium such as body tissue. This absorption of RF power causes a corresponding heating of the medium. The degree of heating or specific absorption rate (SAR) is proportional to the intensity of the field in the medium.

For a resonant half wavelength dipole, the electric field in the medium concentrates near the feed port of the antenna, causing an undesirable SAR hot spot in the medium (body tissue).

Resonance: An antenna design that depends on resonance, such as half-wavelength dipole antenna, suffers losses when present in a lossy medium such as body tissue. The result is this type of antenna has poor power reception due to damping by the lossy medium.

Properties of medium: A single dipole antenna is ideally designed to have a total conductor length of one half of the electromagnetic wavelength in the medium. However, in the body, the media (tissues) surrounding the antenna often have different material properties. In particular, the permittivities of tissues in the body vary over a wide range, meaning the electromagnetic wavelength also varies widely. Further, the effective permittivity of the heterogenous tissues can vary dramatically with small displacements of the antenna. Thus, it is seldom possible to predict the appropriate antenna length for a half-wavelength dipole for a given implantation scenario.

Interference: A half-wavelength dipole antenna is subject to field interference when in proximity to an electrically long wire, a conductor that interacts with electric fields. For example, a user (patient) can have another implanted device in proximity to the Receiver Array such as fixation hardware, a device with an implanted cable, or another Receiver Array. Any electrically long conductor in proximity to a half-wave dipole field shorts the field in the vicinity of the antenna port. This phenomenon greatly reduces the power transmission to the half-wavelength dipole.

RF power requirements: An implantable pulse generator on the market uses energy supplied via RF power from an external transmitter. The implantable pulse generator of this system does not use stored energy for generation of the electrical impulses that it delivers to tissue. The timing of the electrical impulses is directly driven by the timing of the RF bursts, meaning the system requires each RF burst from the external transmitter to deliver the entirety of the energy needed for a given electrical impulse.

The consequence of this existing system architecture is the system requires relatively high-power RF bursts in order to operate the implantable pulse generator. To generate such high-power RF bursts, the external transmitter is by necessity relatively complex, large, heavy, and expensive. The external transmitter produces high RF emissions near the limit of compliance to RF emissions regulations.

### SUMMARY OF THE INVENTION

A medical implantable apparatus device is disclosed, herein denoted as a percutaneous implantable pulse generator (pIPG). The device is considered percutaneous because it is implanted within the body by placement through a small cannula or needle. The outer diameter of the device is limited in that regard to 1.7 mm or smaller.

The medical apparatus of the present invention provides a means of on-board power storage in order for the device to provide therapy for various neurological disorders, including by not limited to urinary urge incontinence, by way of electrical stimulation of nerve fibers in the sacral plexus, tibial, or pudendal nerve bundles, to treat various disorders associated with prolonged symptoms resulting from erectile dysfunction and other sexual dysfunctions, as a therapy to treat chronic pain, and or prevent or treat, or provide power to sensors that are monitoring a variety of other modalities that may present in subjects.

In one or more aspects, a method, system, and the medical apparatus is a fully implantable device containing pulse generating electronics and power storage that can be introduced percutaneously to tissue through a standard sized needle introducer.

Stimulation and control parameters are fully adjustable through the external transmitter to levels that are considered safe and efficacious while being in contact with biological tissue in a charged-balanced approach and with waveform conditioning to provide minimal discomfort. Different neural tissue responses differently to different stimulation waveforms through the recruitment of different nerve fiber types. Some neural tissue response to low frequency stimulation (5 Hz to 100 Hz), which may have an excitatory effect, while higher frequencies, (100 Hz to 20,000 Hz) may result in an inhibitory effect, with a reduction or blocking of neural activity.

In one or more aspects of the disclosure, the medical apparatus may comprise of: a multitude of distinct receiver antenna elements, electronics for pulse generation, power storage, and directly attached electrodes.

These and other aspects are described herein.

### BRIEF DESCRIPTION OF DRAWINGS

FIG. 1 shows a high-level diagram of an example of the medical apparatus implantable device including an external transmitter 10 for transmitting RF input signals 12 to a pIPG 20. The external transmitter 10 may generate a first input signal 30 that powers the pIPG 20. The pIPG 20 receives the signal 30 at the receiver array and converts the power to stored energy. In one embodiment, the energy is stored locally in each receiver element internal to the receiver array 21. In an alternative embodiment, the energy is stored in a central energy storage bank 22 in the pIPG 20. The stored energy 22 is harnessed by the pulse generator 23 to generate electrical impulses that are conveyed to tissue contacts via the signal delivery 24 circuitry. The external transmitter 10 also generates a second input signal 30 that conveys data to the pIPG. The data includes control parameters 12 for the pIPG 20.

FIG. 2 depicts a receiver element 21 to receive and process an electromagnetic first input signal. In this embodiment, the receiver element converts received RF power 26 to rectified power 25 that is output to a power processor electronics 22 in the pIPG 20.

FIG. 3 depicts a receiver element 21 to receive and process an electromagnetic first input signal. In this embodiment, the receiver element 21 may convert the received RF power 22 and store the energy within a local energy storage 27 bank. In one embodiment, the receiver element 21 outputs direct-current (DC) power 28 from the local energy storage 27 bank to other circuits in the pIPG 20.

FIG. 4 depicts a receiver element 21 to receive and process 29 an electromagnetic second input signal 26. In this embodiment, the receiver element 21 may extract data from the signal and output the data 30 to another circuit in the pIPG.

FIG. 5 depicts a receiver array in a pIPG to receive electromagnetic input signals. The receiver array is comprised of a multiplicity of receiver elements 31. In this embodiment, the receiver elements are longitudinally aligned.

FIG. 6 shows an alternative embodiment of the receiver array in which the Receiver Elements 33 are orthogonally aligned. Orthogonal alignment may apply to doublets of elements arranged in a plane, or it may apply for example to triplets in a three-dimensional volume.

FIG. 7A to FIG. 7C shows plots of simulated electric-field contours resulting from RF transmission to a receiver in a medium. The medium properties were defined to mimic an aggregate of body tissue 35. A transmitting antenna was positioned at the upper surface of the medium, and a receiving device was positioned within the medium to receive the RF power. The FIG. 7B plot shows the magnitude of the electric field when the receiving element within the pIPG was a single dipole antenna, and the FIG. 7C plot shows the result when the receiving elements were organized as a receiver array of electrically short receiving elements. The areas of highest electric field in the medium correspond to zones of greatest heat generation in the medium. The receiver array harvests more power along its length compared to a dipole antenna of the same length. Correspondingly, the receiver array reduces the SAR (heating) of the medium.

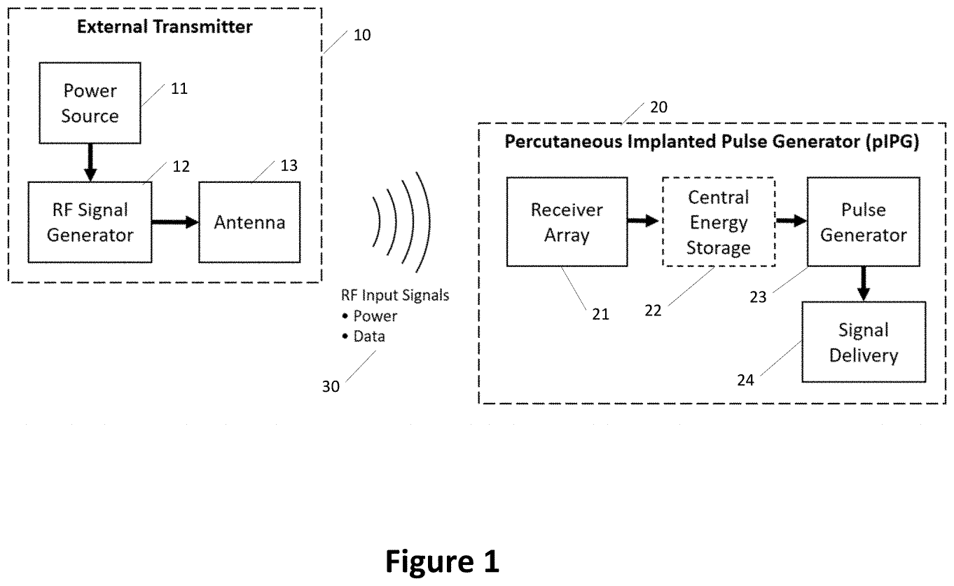

### DETAILED DESCRIPTION OF THE PREFERRED EMBODIMENT

The following descriptive paragraphs detail the contemplation of the invention based on the general principles and scope herein.

The medical apparatus device may include an external transmitter 10 for transmitting electromagnetic RF input signals 30 to a pIPG 20. In one embodiment, the pIPG 20 may be designed to be located at a shallow depth within the medium (tissue) in order to best receive the RF input signals 30 via non-radiative, reactive, near-field coupling. In an alternative embodiment, the pIPG 20 may be designed to be located at a moderate depth within the medium in order to best receive the RF input signals 30 via radiative, non-reactive, near-field coupling. In another alternative embodiment, the pIPG 20 may be designed to be located at a greater depth within the medium in order to best receive the RF input signals 30 via radiative, far-field reception.

A medical apparatus device such as the pIPG may for example deliver therapy to a patient and/or sense physiological measurands. In one implementation, the pIPG may be a neurostimulator device with one or more contacts for delivering electrical impulses to tissue. The pIPG is an enclosure shaped and configured for implantation into the body tissue of a user (patient).

The design includes a receiver array of electrically short receiver elements 31, 33. There are several advantages achieved with this new architecture.

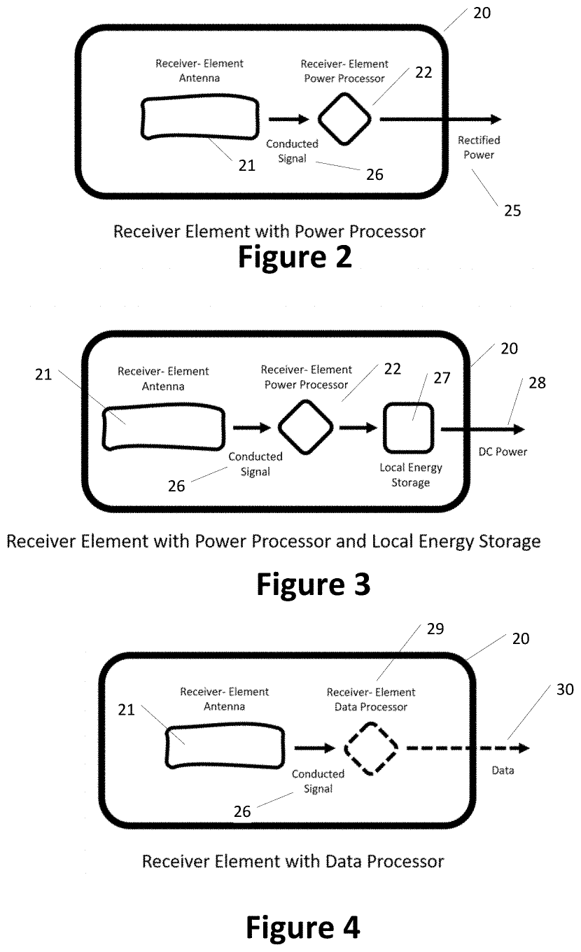

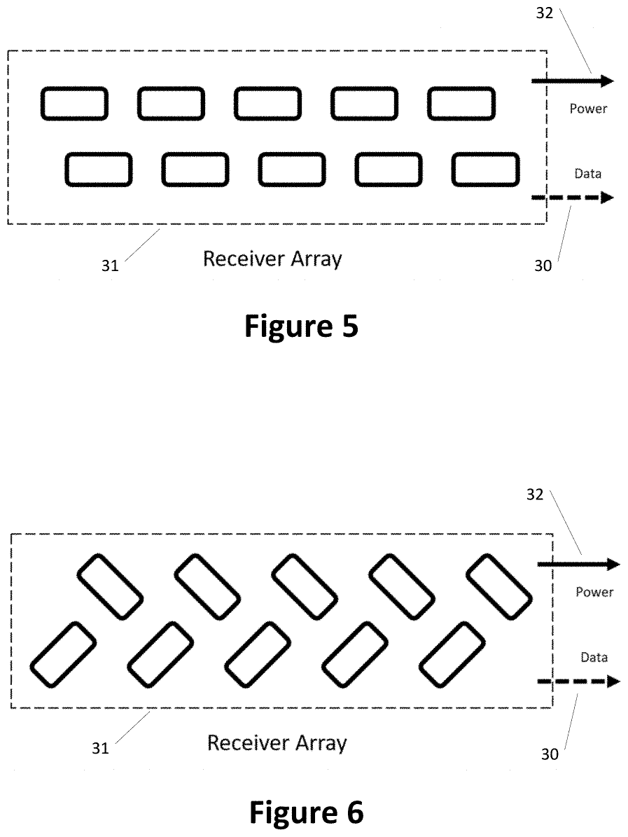

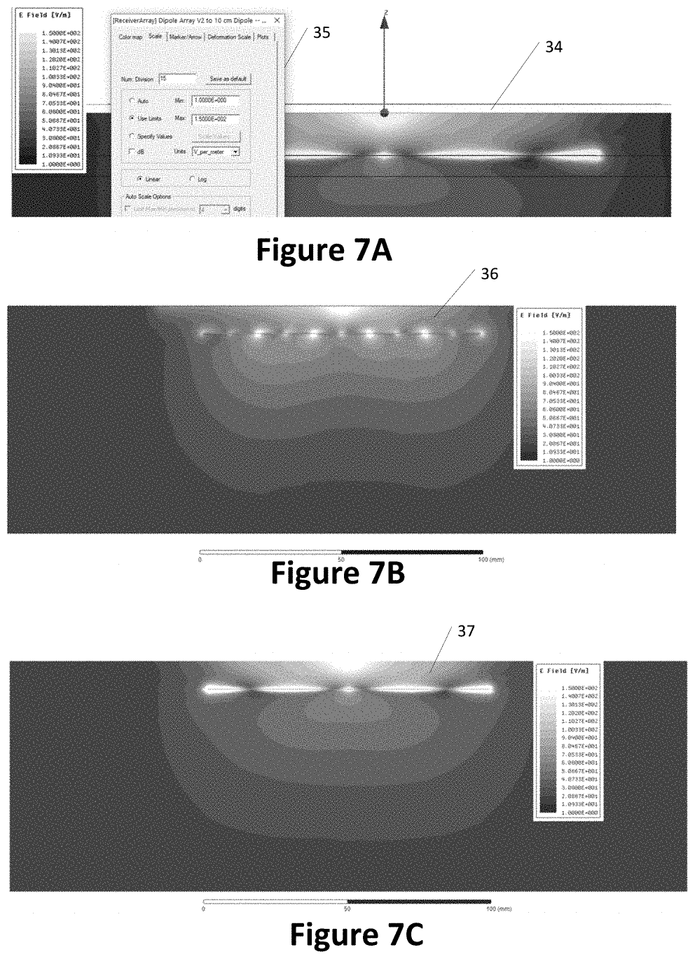

The new system allows the pIPG 20 to receive RF energy from the external transmitter 10 over longer time scales due to having the ability to store the energy on board 22, as shown in FIG. 2 through FIG. 4. In one implementation, a low power, continuous-wave RF input signal 30 can charge the storage system. Once the storage bank 22 in the pIPG 20 is sufficiently charged, the accumulated energy may be discharged or delivered at controlled intervals to a load. For example, the pIPG may deliver an electrical impulse to tissue.

In another implementation, intermittent RF bursts can charge the storage system, wherein the timing of the RF bursts can be independent of the timing of the electrical impulses generated by the pIPG.

This design allows for powering the pIPG 20 using lower peak RF power levels, which in turn allows the external transmitter 10 to operate at lower RF peak power. There are several advantages as a result. The external transmitter 10 can be simpler, smaller, lighter, and less expensive. The external transmitter 10 of the new system produces much lower RF emissions, giving a wide margin for compliance to RF-emissions regulations.

The pIPG 20 houses a receiver array 21 that is composed of two or more receiving elements. A given receiver element is comprised of an electrically small antenna and a processor circuit connected to the receiver element (FIG. 2, 3, 4) antenna port physically on the same substrate. A given receiver element receives a portion of the power of radiofrequency input signals 30 from an external transmitter 10. Electrically small means the physical dimension of a given antenna is a small fraction of the wavelength (lambda) of a radiofrequency input signal in the medium (for example, a tissue medium). In one implementation, a given receiver element is smaller than 1/10^{th} of the wavelength.

Receiver elements may be connected to one another in various configurations all on the same type of flexible circuit board substrate, usually made from a type of plastic called a polyimide. In one embodiment of the receiver array, the distributed receiver elements are connected to one another directly. In an alternative embodiment of the receiver array, the distributed receiver elements are isolated from one another both physically and electrically.

The antenna ports of receiver elements may be interconnected to optimize RF voltages and currents. In one embodiment, the antenna ports are connected in series to maximize the RF peak voltage of the receiver array. In an alternative embodiment, the antenna ports are connected in parallel to maximize the RF peak current of the receiver array. Alternative connection patterns may be employed.

Similarly, the outputs of receiver elements may be interconnected. In one embodiment, the outputs are connected in series to maximize the output voltage of the receiver Array. In an alternative embodiment, the outputs are connected in parallel to maximize the output current of the receiver array. Alternative connection patterns may be employed.

Angle relative to face of transmitting antenna: This design is angle independent by virtue of each element being substantially shorter than the wavelength in the medium. This greatly reduces the phase sensitivity of the array, meaning when the receiver array is angled away from the face of the transmitting antenna, the receiver array as a whole is not subject to destructive interference.

Angle relative to polarization of transmitting antenna (Azimuth angle): In some embodiments, a receiver array may be configured such that its receiver elements have different polarizations relative to each other, with the result that the receiver array is non-polarized. For example, the elements in the array may be arranged orthogonally, making the receiver array capable of receiving RF input signals when rotated to any azimuth angle.

Number of extraction circuits: A receiver array of electrically short receiver elements may receive RF input signals on all of the receiver elements, or it can receive RF input signals on only a subset of its receiver elements. The system is designed to operate regardless of the number of elements receiving signal, so long as the combination of element outputs is sufficient to operate the pIPG.

Longitudinal alignment relative to transmitting antenna: Since the Receiver Array can receive RF input signals on a subset of its Receiver Elements, the Receiver Array has negligible sensitivity to longitudinal misalignment with the external transmitting antenna. Optimal transmission is achieved over a wide range of longitudinal displacement of the receiver array relative to the center of the external transmitting antenna.

Size of antenna aperture: Further, since the receiver array can receive RF input signals on a subset of its receiver elements, this characteristic allows for the array to be arbitrarily increased in size, which effectively extends the aperture of the array. For example, the aperture size of the receiver array may be made substantially greater than the aperture size of a half-wavelength dipole antenna.

Geometrical linearity of antenna: The receiver array is free of the need to maintain geometrical linearity. A receiver array composed of multiple short receiver elements can follow an arbitrarily curved path in the medium, because each receiver element harvests electric field energy independent from the other elements.

Specific absorption rate (SAR): Since the degree of heating or specific absorption rate (SAR) is proportional to the intensity of the field in the medium, it is advantageous to reduce the intensity of the field by increasing the number of receiving elements. For the receiver array, the field is not concentrated near any one feed port, thus the receiver array prevents undesirable SAR hot spots in the medium. (body tissue).

Resonance: A receiver array of electrically short receiver elements is not reliant on resonance. Electric field oscillation is minimized due to very short conductor lengths of the elements, and waves need not propagate very far along a conductor before being harvested by a receiver element. Since the wave propagation distance and oscillation is minimized, the losses associated with propagation and oscillation are also minimized. The result is the sum of outputs from all receiver elements in the distributed receiver array is equivalent or higher than the output of a resonant half-wave dipole.

Properties of medium: The media (tissue) surrounding the receiver array does not affect the power transmission to the receiver array, because each receiver element is electrically short and non-resonant. Since a given element is non-resonant, the electromagnetic wavelength in the medium does not affect the element. This means the permittivities of tissues surrounding the receiver array can vary over a wide range without changing the performance of the receiver array. This also implies the overall size of the receiver array is not tied to the electromagnetic wavelength in the medium, and consequently it is not necessary to predict the wavelength for a given implantation scenario.

Interference: A receiver array experiences less field interference when in proximity to an electrically long connectivity trace. Although an electrically long conductor in proximity to a receiver array shorts the field in the vicinity of a single receiver element, the field is redistributed to another element in the array. This aspect of the array design allows power transmission to the receiver array in spite of proximity to a long embedded connectivity trace.

RF power requirements: This invention includes an energy storage system in the pIPG that can be charged at an arbitrary rate independent of the timing of the electrical impulses generated by the pIPG. The new design obviates the need to supply high-power RF bursts to the pIPG from the external transmitter. The energy of an electrical impulse can be accumulated over time, and it need not be supplied by a single, high-power RF burst. For example, the RF energy may be harvested in the interval between electrical impulses.

A processor circuit herein means a circuit to convert RF input signals to either useful output power or data (information). The power processor internal to a receiver element harvests RF power from the First Input Signal and conducts the rectified power to either a local energy storage bank internal to the receiver element or directly to an output of the receiver element. The data processor internal to a receiver element receives the second input signal and extracts data, which is conveyed to an output of the receiver element.

An external transmitter is an apparatus external to the body of the user (patient). The external transmitter generates radiofrequency signals and transmits them wirelessly into the body of the user (patient) via an external antenna. These input signals reach the pIPG receiver array, which may use the input signals for operation.

The external antenna of the external transmitter may create electromagnetic fields in the medium (tissue). Depending on the properties of the medium, these electromagnetic fields may have field zones described as 1) non-radiative, reactive, near-field, 2) radiative, non-reactive, near-field, or 3) radiative, far-field. In practice, the pIPG may be implanted in a medium at any of these field zones. The configuration of the pIPG may be designed to attain its best reception of input signals based upon the specific field zone in which it may be located.

In one embodiment, the configuration of the pIPG may be adjusted to attain its best signal reception in a given field zone. In an alternative embodiment, the pIPG may be configured to attain sufficient signal reception across any of the field zones but at the expense of attaining best reception in a given zone. In the various embodiments, the configuration of the pIPG may be altered, for example, by changing the lengths of the receiver elements, changing the number of the receiver elements, changing the input connections of the receiver elements, changing the output connections of the receiver elements, and changing the processor design of the receiver elements.

The signals generated by the external transmitter in various embodiments may have different radio frequencies, different power levels, and different duty cycles. In one embodiment, a first input signal may be purposefully configured to convey operational energy to the pIPG. In one embodiment, a second input signal may be configured to convey data to the pIPG.

A first input signal may convey energy to the pIPG that is stored and used for operation of the pIPG. In one embodiment, multiple local energy storage banks internal to the receiver elements form a distributed storage system. In an alternative embodiment, each receiver element may output its rectified signal to a central energy storage bank in the pIPG.

A second input signal may convey data to the pIPG. A given receiver element within the receiver array may extract and output the data signal to a controller within the pIPG. For example, the data may include parameter-setting attributes to change an operation mode of the pIPG.

Generally, the first input signal does not have characteristics suitable for direct conversion into electrical impulses suitable for neural modulation. The first input signal may for example be continuous wave (CW), or it could be pulsed with a purposefully designed pulse envelope for optimal charging of the energy storage banks in pIPG. Further, the pulse timing and duration of the first input signal may have no correlation to the pattern and timing of electrical impulses that are subsequently created by the pIPG.

In the pIPG, a storage device receives rectified electrical current and accumulates charge for later use by circuits in the pIPG. In one embodiment, the storage device is one or more capacitors. In an alternative embodiment, the storage device is an electrochemical cell or battery. In another embodiment, a hybrid system uses both capacitor- and battery technology. In one embodiment of the pIPG, a central energy storage device is located in one location within the enclosure, and a charging circuit connects the storage device to multiple receiver elements of the receiver array. In an alternative embodiment of the pIPG, Local energy storage devices may be located internally within multiple receiver elements of the receiver array. In this embodiment, the energy storage in the pIPG is a distributed network, and the outputs of the local energy storage devices are conducted to other circuits within the pIPG.

The pIPG uses the stored energy from the first input signal to operate. A circuit in the pIPG processes data conveyed in the second input signal. The operational state of the pIPG may be configured by the data.

A circuit in the pIPG is connected to tissue contacts and is designed to generate electrical impulses from the stored energy. A given electrical impulse or pattern of impulses created by the pIPG may be tailored to influence excitable tissue in the body. Tissue contacts may be constructed from material suitable for long-term implantation in body tissue, and the size and shape of the contacts may be chosen to have minimal irritation to tissue and to pass electrical charge to tissue while maintaining safe current densities.

Other electrode arrangements will be apparent to those skilled in the art. Many of these arrangements may be modified to provide various configurations of the receiver antenna array layouts, and for electrode arrangements that may intend to fall within the scope of the present invention.

A number of implementations have been described. Nevertheless, it will be understood that various modifications may be made. Accordingly, other implementations are within the scope of the following claims.

Although the present invention has been particularly described with reference to embodiments thereof, various changes, modifications and substitutes are intended within the form and details thereof.

Many modifications and variations of the present invention are possible in light of the above teachings. It is therefore to be understood that, within the scope of the appended claims, the invention may be practiced otherwise them as specifically described.

What is claimed is:

1. A medical apparatus comprising:
A tubular shaped enclosure configured for implantation into a tissue medium;
a receiver array with a multitude of receiver elements housed within the enclosure attached to the associated electronics via a flexible circuit board construction,
wherein the receiver array is configured to receive one or more electromagnetic input signals of a combination of both power and data from an external transmitter via non-inductive coupling energy transfer,
wherein the receiver array is composed of multiple receiver elements, wherein each receiver element within the receiver array includes an electrically small antenna and one or more processor circuits connected to the port of the antenna on the same physical substrate,
wherein the receiver array and associated flexible circuit board are directly attached to two or more electrodes that are in direct contact with biological tissue for the purpose of transmitting stimulation pulses to tissue.

2. The medical apparatus of claim 1 in which a self-contained power source is embedded on the same physical flex board substrate as the receiver array, and can range from a primary battery, a rechargeable battery, a super capacitor, a nuclear battery, a mechanical resonator, a thermally-powered energy source, an optically powered energy source, a bioenergy source, or other tissue transfer energy source.

3. The medical apparatus of claim 1 in which one or more receiver elements of the receiver array attached to the flexible circuit board construction that
contains a self-contained power processing circuit that converts energy into rectified power wherein the electromagnetic power of a first input signal is received from an external transmitter.

4. The medical apparatus of claim 1 in which a circuit
is connected to one or more receiver elements of the receiver array, conducts rectified power from one or more power processor circuits, and connects to a central energy storage device.

5. The medical apparatus of claim 1 in which one or more receiver elements of the receiver array
contain a local energy storage device,
wherein the local energy storage device stores energy from rectified power conducted from the receiver element power processor circuit,
wherein the local energy storage device is further connected to an output signal of the receiver element.

6. The medical apparatus of claim 1 in which a circuit
connects to electrode contacts on an integrated flexible substrate,
generates electrical impulses to the electrode contacts that in contact with biological tissue.

7. The medical apparatus of claim 1 in which
one or more receiver elements of the receiver array, which
contain a data processor circuit that extracts data from a second input signal received from an external transmitter,
is further connected to an output of the receiver element;

8. The medical apparatus of claim 1 in which
a controller circuit is
connected to one or more energy storage devices,
receives data from one or more data processor circuits,
connected to tissue contacts,
generates electrical impulses.

9. The medical apparatus of claim 1 in which
the medical apparatus is configured to attain enhanced reception of input signals when a portion of its receiver array is located in the non-radiative, reactive, near-field zone of an antenna of an external transmitter.

10. The medical apparatus of claim 1 in which
the medical apparatus is configured to attain enhanced reception of the input signals when a portion of its receiver array is located in the radiative, non-reactive, near-field zone of an antenna of an external transmitter.

11. The medical apparatus of claim 1 in which
the medical, apparatus is configured to attain enhanced reception of the input signals when a portion of its receiver array is located in the radiative, far-field zone of an antenna of an external transmitter.

12. The medical apparatus of claim 1 in which
the medical apparatus is introduced to tissue through an introducer of no greater than 1.7 mm in diameter.

13. The medical apparatus of claim 1 which includes receiver element orientations aligned with the receiver and transmitter total electric field (complex magnitude), placed approximate to the center of the receiver and transmitter electric field.
